(19) United States
(12) Patent Application Publication
(10) Pub. No.: US 2016/0106378 A1
(43) Pub. Date: Apr. 21, 2016

(54) SYSTEM AND METHOD FOR DETECTING AN ARRHYTHMIC CARDIAC EVENT FROM A CARDIAC SIGNAL

(71) Applicant: XEROX CORPORATION, Norwalk, CT (US)

(72) Inventors: Survi KYAL, Rochester, NY (US); Lalit Keshav MESTHA, Fairport, NY (US); Beilei XU, Penfield, NY (US)

(21) Appl. No.: 14/519,607

(22) Filed: Oct. 21, 2014

(51) Int. Cl. A61B 5/00; A61B 5/11; A61B 8/02; A61B 5/0476; A61B 5/0488; A61B 7/00; A61B 5/0402; A61B 5/0205

(52) U.S. Cl. CPC A61B 5/7282; A61B 5/7257; A61B 5/7267; A61B 5/726; A61B 5/0402; A61B 5/1102; A61B 5/0205; A61B 5/0476; A61B 5/0488; A61B 7/00; A61B 8/02; A61B 5/0077; A61B 5/746; A61B 5/747; A61B 5/742; A61B 5/0004; A61B 5/6898; A61B 5/0022; A61B 5/02405

(57) ABSTRACT

What is disclosed is a system and method for detecting an arrhythmic or non-arrhythmic event from a cardiac signal obtained from a subject. In one embodiment, a plurality of different cardiac signals are received and are transformed into frequency domain signals which, in turn, are changed such that a dominant frequency in each of the signals is substantially aligned to form a matrix of feature vectors. The feature matrix is used to train a classifier. A cardiac signal from the subject is received and transformed to a frequency domain signal. The frequency domain signal is changed such that a dominant is substantially aligned with a dominant frequency of signals used to train the classifier. The subject's frequency domain signal is provided as a new feature vector to the classifier. The classifier uses the new feature vector to classify the subject as having an arrhythmic or a non-arrhythmic event.

### TECHNICAL FIELD

The present invention is directed to systems and methods for detecting an arrhythmic cardiac event from a cardiac signal obtained from a subject being monitored for cardiac function.

### BACKGROUND

Early detection of cardiac arrhythmias can be critical for patient recovery. Increasingly sophisticated systems and methods for monitoring for various cardiac events are needed to improve diagnosis and treatment. The present invention is directed toward detecting an arrhythmic cardiac event from a cardiac signal obtained from a subject being monitored for cardiac function.

### INCORPORATED REFERENCES

The following U.S. patents, U.S. patent applications, and Publications are incorporated herein in their entirety by reference.

“Identifying A Type Of Cardiac Event From A Cardiac Signal Segment”, U.S. patent application Ser. No. 14/492,948, by Xu et al. (Attorney Docket No. 20140386 US01).

“Estimating Cardiac Pulse Recovery From Multi-Channel Source Data Via Constrained Source Separation”, U.S. patent application Ser. No. 13/247,683, by Mestha et al.

“Continuous Cardiac Pulse Rate Estimation From Multi-Channel Source Video Data”, U.S. patent application Ser. No. 13/528,307, by Kyal et al.

“Continuous Cardiac Pulse Rate Estimation From Multi-Channel Source Video Data With Mid-Point Stitching”, U.S. patent application Ser. No. 13/871,728, by Kyal et al.

“Continuous Cardiac Signal Generation From A Video Of A Subject Being Monitored For Cardiac Function”, U.S. patent application Ser. No. 13/871,766, by Kyal et al.

“Determining Cardiac Arrhythmia From A Video Of A Subject Being Monitored For Cardiac Function”, U.S. patent application Ser. No. 13/532,128, by Mestha et al.

### BRIEF SUMMARY

What is disclosed is a system and method for detecting an arrhythmic cardiac event from a cardiac signal obtained from a subject being monitored for cardiac function. One embodiment of the present method involves the following. First, a plurality of different cardiac signals are received. The cardiac signals can be received from a plurality of cardiac function monitoring devices or may be a videoplethysmographic signal obtained from processing overlapping batches of video image frames of the subject where a plethysmographic signal can be registered by at least one channel of the video device used to capture those video images. The received cardiac signals are then transformed into frequency domain signals using, for example, a Fast Fourier Transform (FFT) or a Discrete Cosine Transform (DCT) method. The frequency domain signals are then changed such that dominant frequencies in each of the signals are substantially aligned. This alignment forms a matrix of feature vectors, whose dimension is then reduced by performing Principal Component Analysis. The reduced matrix is an eigen feature matrix. The eigen feature matrix is used to train a classifier. The classifier can be, for instance, a Support Vector Machine (SVM) classifier, or a neural network, as are commonly understood. Once the classifier has been trained, one or more cardiac signals from a subject are received. The subject's cardiac signal is transformed to a frequency domain signal which is changed in a manner such that a dominant frequency in that cardiac signal are substantially aligned with a dominant frequency of the signals used to train the classifier. If the subject's frequency domain signal has a frequency bandwidth which is different than that of the signals used to train the classifier then the subject's frequency domain signal is changed to have a same frequency bandwidth and a same signal length as those training signals. The subject's frequency domain signal is then provided as a new feature vector to the classifier. The classifier then uses the new feature vector to classify the subject as having one of: an arrhythmic event, and a non-arrhythmic event. Many features and advantages of the present method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a video image device capturing video of a subject;

FIG. 2 shows the subject of FIG. 1 laying in an upright position with a plurality of contact-based electrodes attached to the chest where cardiac signals are actively being captured by an electrocardiographic device;

FIG. 3 is a flow diagram which illustrates one example embodiment of the present method for detecting a cardiac event from a cardiac signal of a subject;

FIG. 4 is a continuation of the flow diagram of FIG. 3 with flow processing continuing with respect to node A;

FIG. 5 illustrates a block diagram of one example signal processing system 500 for detecting an arrhythmic event from a cardiac signal obtained from a subject in accordance with the embodiment described with respect to the flow diagrams of FIGS. 3 and 4; and

FIG. 6 shows the error rates during training calculated for both ECG Automatic and ECG Manual using SDRR, RMSSD, pNN50, SD1 and SD2.

### DETAILED DESCRIPTION

What is disclosed is a system and method for detecting an arrhythmic cardiac event from a cardiac signal obtained from a subject being monitored for cardiac function.

### Non-Limiting Definitions

A “subject” refers to a living being. Although the term “person” or “patient” may be used throughout this disclosure, it should be appreciated that the subject may be something other than a human such as, for example, a primate. Therefore, the use of such terms is not to be viewed as limiting the scope of the appended claims strictly to humans.

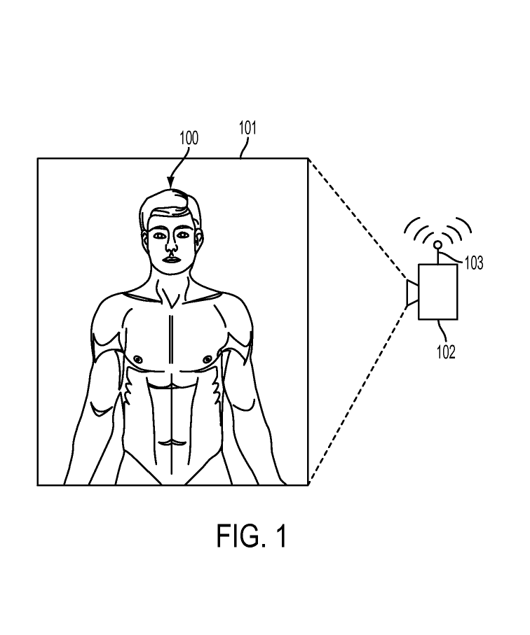

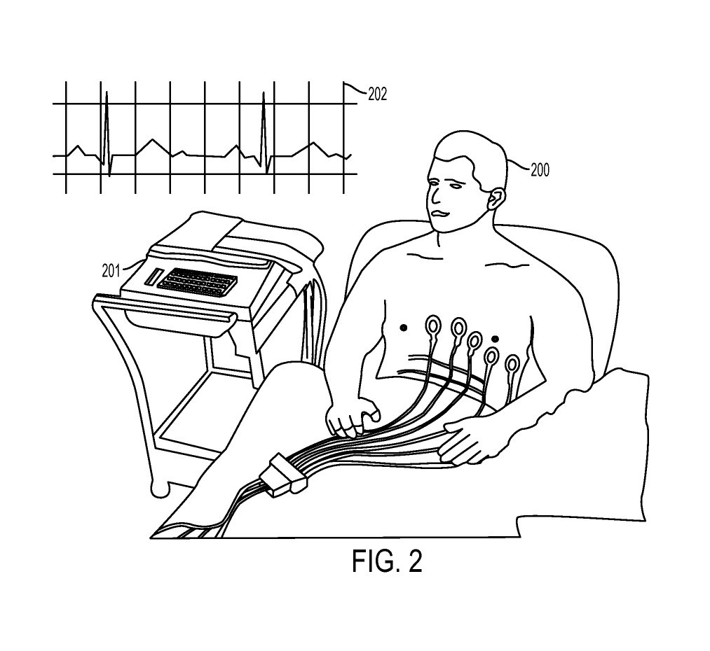

A “cardiac signal” is a signal which relates to the function of the subject's heart. Cardiac signals can be, for instance, a videoplethysmographic (VPG) signal extracted from a time-series signal obtained from processing overlapping batches of image frames captured by an imaging device such as, a video camera, a RGB camera, a multi-spectral or hyperspectral imaging system, and a hybrid device comprising any combination thereof. Such imaging devices typically have a plurality of outputs where the captured images are obtained on a per-channel basis. FIG. 1 shows one example embodiment of a video imaging device 102 actively capturing video 101 of a subject 100. The video image frames of the subject are communicated to a remote computing device via a wireless transmissive element 103, shown as an antenna, where the image frames of the video are processed in partially overlapping batches to obtain a time-series signal. VPG signals are extracted from the time-series signals. Methods for obtaining a time-series signal from video image frames and for extracting VPG signals are disclosed in several of the incorporated references. A cardiac signal can also be received from specialized medical instrumentation such as, for instance, an electrocardiographic device, an echocardiographic device, an electromyographic device, an electroencephalographic device, a phonocardiographic device, and a ballistocardiographic device. FIG. 2 shows a patient 200 laying in an upright position with a plurality of contact-based electrodes attached to the chest where cardiac signals are actively being captured by an electrocardiographic device 201 on a cart. The electrocardiographic device receives cardiac signals from the patient. In one embodiment, the patient's cardiac signal takes the form of an electrocardiogram 202. Various imaging devices and specialized medical instrumentation for obtaining cardiac signals may incorporate memory, a storage device, and one or more microprocessors executing machine readable program instructions. The cardiac signal can be normalized to a frequency of a normalized heartbeat. A length of a cardiac signal can be a single cardiac cycle or a multiplicity of cardiac cycles. The cardiac cycles can be normalized cardiac cycles.

A “feature vector” contains features of interest obtained by analyzing the cardiac signal. A feature of interest may be one or more aspects of, for instance, a frequency domain version of the cardiac signal. In addition to this, features of interest may be one or more higher order statistical quantities obtained by analyzing a set of peak-to-peak intervals of a cardiac signal with respect to any of: a mean, standard deviation, skewness, and kurtosis. Features of interest may be one or more heart rate variability metrics obtained by analyzing a cardiac signal with respect to any of: a Standard Deviation of RR Intervals (SDRR), Root Mean Square of Successive RR Difference (RMSSD), Proportion of NN or RR interval exceeding 50 milliseconds (pNN50), Shannon Entropy (ShE), Standard Deviation 1 (SD1), Standard Deviation 2 (SD2), Pulse Harmonic Strength (PHS), and Normalized Pulse Harmonic Strength (NPHS). Patient information and medical histories may further be associated with various cardiac signals and features of interest.

A “matrix of feature vectors” is a dimensionality reduced matrix comprising an eigen feature matrix. In one embodiment, the feature vector is obtained by having performed a Principal Component Analysis (PCA) method. The matrix of features vectors is used to train a classifier.

A “classifier” is an artificial intelligence system which functions to map feature spaces to labels which define each space. Once trained, the classifier receives a new set of features for an unclassified event and assigns a label to those features. Classifiers can take any of a variety of forms including a Support Vector Machine (SVM), a neural network, a Bayesian network, a Logistic regression, Naïve Bayes, Randomized Forests, Decision Trees and Boosted Decision Trees, K-nearest neighbor, and a Restricted Boltzmann Machine (RBM), as are understood in the machine learning arts. For an in-depth discussion, the reader is directed to any of a wide variety of texts on classifiers, including: “Foundations of Machine Learning”, by Mehryar Mohri, Afshin Rostamizadeh, Ameet Talwalkar, MIT Press (2012), ISBN-13: 978-0262018258, and “Design and Analysis of Learning Classifier Systems: A Probabilistic Approach”, by Jan Drugowitsch, Springer (2008), ISBN-13: 978-3540798651, both of which are incorporated herein in their entirety by reference. The classifier uses the subject's feature vector to classify the subject as having an arrhythmic or non-arrhythmic event.

An “arrhythmic event” refers to a cardiac arrhythmia, (also known as cardiac dysrhythmia), which is an irregular heartbeat. One common cardiac arrhythmia is atrial fibrillation. Other arrhythmias include ventricular tachycardia, sinus tachycardia, sinus bradycardia, and ventricular premature contraction, as are generally understood in the medical arts. The reader is referred to any of a variety of medical texts for a thorough discussion on the different cardiac arrhythmias which comprise an arrhythmic event.

A “non-arrhythmic event” refers to the heart's normal sinus rhythm (SR).

A “frequency domain signal” is a signal obtained by performing any of: a non-parametric spectral density estimation on a cardiac signal, a parametric spectral density estimation on a cardiac signal, a Fourier Transform, a Wavelet Transform, and/or a Discrete Cosine Transform. Methods for transforming a signal into a frequency domain signal are well established in the signal processing arts.

“Receiving cardiac signals” is intended to be widely construed and includes retrieving, capturing, acquiring, or otherwise obtaining cardiac signals for processing in accordance with the methods disclosed herein. The cardiac signals can be retrieved from a memory or storage of the device used to acquire those signals. Cardiac signals can be retrieved from a media such as a CDROM or DVD, or can be received from a remote device over a network. Cardiac signals may be downloaded from a web-based system or application which makes such signals available for processing.

“Changing frequency domain signals” means to shift, scale, or otherwise modify the frequency domain signals such that a dominant frequency in each of those signals are substantially aligned. In one embodiment, the frequency domain signals are changed to have the same sampling frequency and the same signal length as the signals used to train the classifier. The aligned signals form feature vectors which, in turn, are used to form the matrix of feature vectors.

It should be appreciated that the steps of “transforming”, “changing”, “training”, “providing”, “processing”, and the like, as used herein, include the application of various signal processing and mathematical operations as applied to data and signals, according to any specific context or for any specific purpose. The terms in this Detailed Description and claims include any activity, in hardware or software, having the substantial effect of the mathematical or signal-processing action. It should be appreciated that such steps may be facilitated or otherwise effectuated by a microprocessor executing machine readable program instructions retrieved from a memory or storage device.

### Flow Diagram of One Embodiment

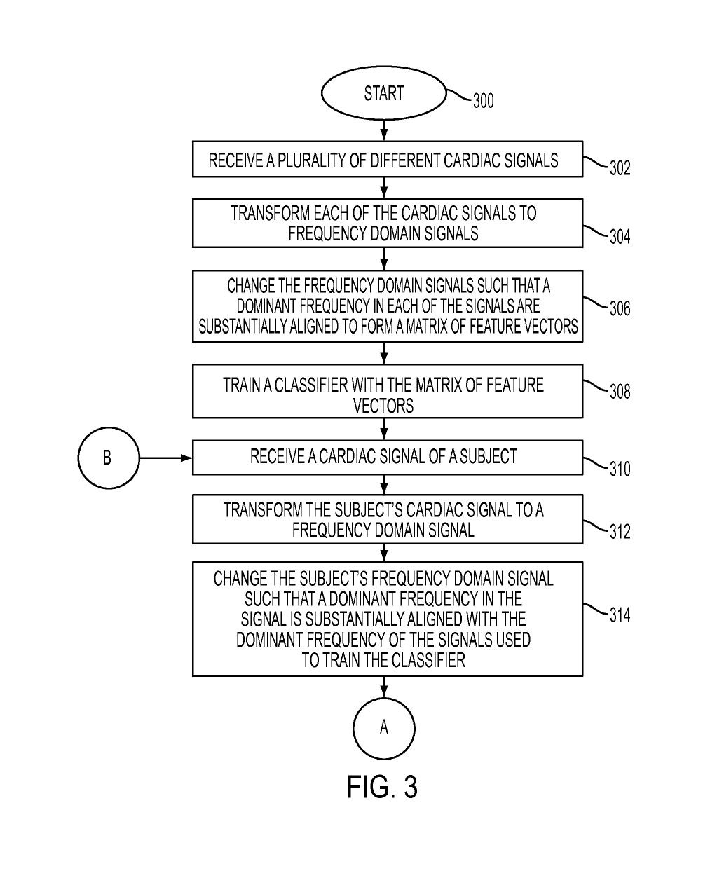

Reference is now being made to the flow diagram of FIG. 3 which illustrates one example embodiment of the present method for detecting a cardiac event from a cardiac signal of a subject. Flow processing begins at step 300 and immediately proceeds to step 302.

At step 302, receive a plurality of different cardiac signals.

At step 304, transform each of the cardiac signals to frequency domain signals.

At step 306, change the frequency domain signals such that a dominant frequency in each of the signals are substantially aligned to form a matrix of feature vectors.

At step 308, train a classifier with the matrix of feature vectors.

At step 310, receive a cardiac signal of a subject. One embodiment of cardiac signals being obtained for a subject is shown in FIG. 3.

At step 312, transform the subject's cardiac signal to a frequency domain signal.

At step 314, change the subject's frequency domain signal such that a dominant frequency in the signal is substantially aligned with the dominant frequency of the signals used to train the classifier.

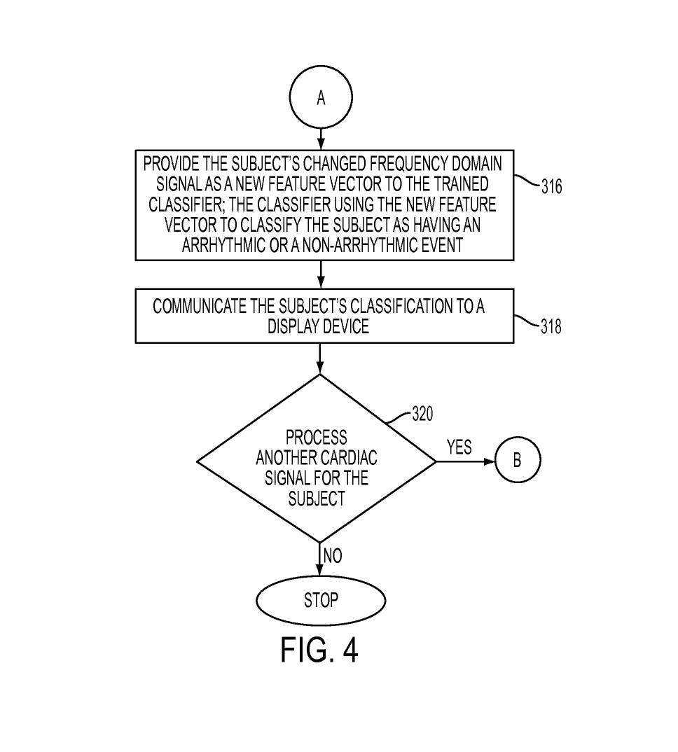

Reference is now being made to the flow diagram of FIG. 4, which is a continuation of the flow diagram of FIG. 3 with flow processing continuing with respect to node A.

At step 316, provide the subject's changed frequency domain signal as a new feature vector to the trained classifier, the classifier using the new feature vector to classify the subject as having one of: an arrhythmic event, and a non-arrhythmic event.

At step 318, communicate the subject's classification to a display device. An alert signal may be initiated and/or a signal sent to a medical professional.

At step 320, a determination is made whether to receive and process another cardiac signal from the subject. If so, then processing repeats with respect to node B wherein, at step 310, a next cardiac signal of the subject is received. Processing repeats in a similar manner for this next received cardiac signal. Processing can repeat for each next received cardiac signal on a real-time and on a continuous basis. If, at step 320, no more of the subject's cardiac signals are to be processed then further processing stops.

It should be appreciated that the flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims.

### Block Diagram of Signal Processing System

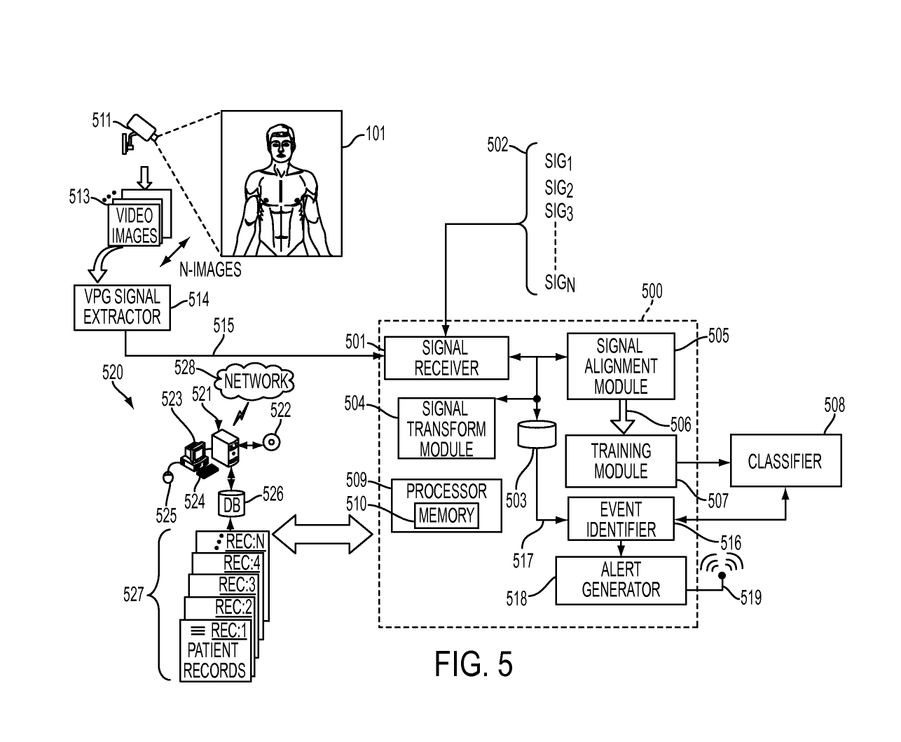

Reference is now being made to FIG. 5 which illustrates a block diagram of one example signal processing system 500 for detecting an arrhythmic event from a cardiac signal obtained from a subject being monitored for cardiac function in accordance with the embodiment described with respect to the flow diagrams of FIGS. 3 and 4.

Signal Receiver 501 acts as a buffer and receives n≧2 cardiac signals (collectively at 502) and stores the received cardiac signals to storage device 503. Signal Receiver 501 may be used for queuing additional signals, as needed including information about the received signals signal length, detected peaks, time/date information, and the like, and may further be configured to also store data, mathematical formulas and other representations to facilitate processing of received cardiac signals in accordance with the teachings hereof. Signal Transform Module 504 retrieves the stored cardiac signals and transforms each of the received cardiac signals to frequency domain signals. The frequency domain signals are then stored to storage device 503. Signal Alignment Module 505 retrieves the frequency domain signals and proceeds to change the frequency domain signals such that a dominant frequency in each of the training signals is substantially aligned. Alternatively, the dimensionality of the aligned signals is reduced. One method for reducing dimensionality is by Principle Component Analysis (PCA). The aligned signals are stored to storage device 503. Module 505 also provides as output a matrix of feature vectors 506 to Training Module 507. The Training Module uses the matrix of feature vectors to train Classifier 508. Although the Classifier is shown as being external to the Signal Processing System 500, in other embodiments, the Classifier is integrated with the other modules and processors of the system 500. Central Processor (CPU) 509 is provided generally to facilitate the functionality of any of the modules of the signal processing system 500. Microprocessor 509, operating alone or in conjunction with other processors, retrieves machine readable program instructions from Memory 510 and is configured to assist or otherwise facilitate the performance of any of the functionality of any of the components of system 500. Processor 509 further effectuates communication between various modules of system 500 and with workstation 521.

Video imaging device 511 acquires streaming video of the subject 100 of FIG. 1. Video image frames (collectively at 513) are communicated to a VPG Signal Extractor 514 which receives batches of image frames and isolates pixels associated with the exposed body region in each of the image frames. The isolated pixels are processed to obtain a time-series signal for each batch in a manner as disclosed in several of the incorporated references. A VPG signal is then extracted from the time-series signal and a cardiac signal 515 for the subject is obtained. The subject's cardiac signal is provided to Signal Receiver 501 which stores the subject's cardiac signal to storage device 503. Signal Transform Module 504 retrieves the subject's cardiac signal from storage device 503 and transforms the cardiac signal to a frequency domain signal in a manner as was done with the received plurality of cardiac signals 502. The frequency domain signal is stored to the storage device. Signal Alignment Module 505 retrieves the subject's frequency domain signal from storage device 503 and proceeds to change the frequency domain signal such that dominant frequencies in that signal are substantially aligned with dominant frequencies of the cardiac signals 502 used to train the Classifier 508. The subject's changed frequency domain signal is stored to storage device 503. Event Identifier Module 516 retrieves the subject's stored changed and aligned frequency domain signal (at 517) from storage and provides the retrieved signal 517 to the Classifier 508 which, once trained, proceeds to classify the subject as having either an arrhythmic event or a non-arrhythmic event. Alert Generator 518 receives the subject's classification and in response to the subject being classified as having an arrhythmia, proceeds to initiate an alert signal to a display device via communication element 519.

Workstation 520 has a computer case 521 which houses various components such as a motherboard with a processor and memory, a network card, a video card, a hard drive capable of reading/writing to machine readable media 522 such as a floppy disk, optical disk, CD-ROM, DVD, magnetic tape, and the like, and other software and hardware needed to perform the functionality of a computer workstation. The workstation further includes a display device 523, such as a CRT, LCD, or touchscreen device, for displaying information, video, distances, clusters, features of interest, computed values, medical information, results, and the like, which are produced or are otherwise generated by any of the block modules of system 500. A user can view any of that information and make a selection from menu options displayed thereon. Keyboard 524 and mouse 525 effectuate a user input or selection as needed. The workstation implements a database 526 wherein records (collectively at 527) are stored, manipulated, and retrieved in response to a query. Such records, in various embodiments, take the form of patient medical history stored in association with information identifying the patient along with medical information. Although the database is shown as an external device, the database may be internal to the workstation mounted, for example, on a hard disk therein.

The workstation has an operating system and other specialized software configured to display alphanumeric values, menus, scroll bars, dials, slideable bars, pull-down options, selectable buttons, and the like, for entering, selecting, modifying, and accepting information needed for training a classifier and using that classifier in accordance with the methods disclosed herein. A user or technician may use the workstation to analyze the cardiac signals, identify features of interest, associate various features of interest with different cardiac events, train the classifier, set various parameters, select cardiac signals for processing, and/or use the workstation to facilitate the functionality of any of the modules and processing units of Signal Processing System 500. User input and selections may be stored/retrieved in either of storage devices 503 and 526. Default settings and initial parameters can also be retrieved from any of these storage devices. A user may adjust various parameters being utilized or dynamically adjust settings in real-time. The alert signal generated may be received and viewed by the workstation 520 and/or communicated to one or more remote devices over network 528.

Although shown as a desktop computer, it should be appreciated that the workstation 520 can be a laptop, mainframe, or a special purpose computer such as an ASIC, circuit, or the like. The embodiment of the workstation is illustrative and may include other functionality known in the arts. Any of the components of the workstation may be placed in communication with any of the modules of system 500 or any devices placed in communication therewith. Moreover, any of the modules of system 500 can be placed in communication with storage devices 503 and 526 and/or computer readable media 522 and may store/retrieve therefrom data, variables, parameters, signals, records, mathematical functions, and machine readable/executable program instructions, as needed to perform their intended functions. Moreover, any of the modules of system 500 may be placed in communication with one or more remote devices over network 528.

It should be appreciated that some or all of the functionality performed by any of the modules or processing units of the system of FIG. 5 can be performed, in whole or in part, by the workstation 520. The embodiment shown is illustrative and should not be viewed as limiting the scope of the appended claims strictly to that configuration. Various modules may designate one or more components which may, in turn, comprise software and/or hardware designed to perform the intended function.

### Performance Results

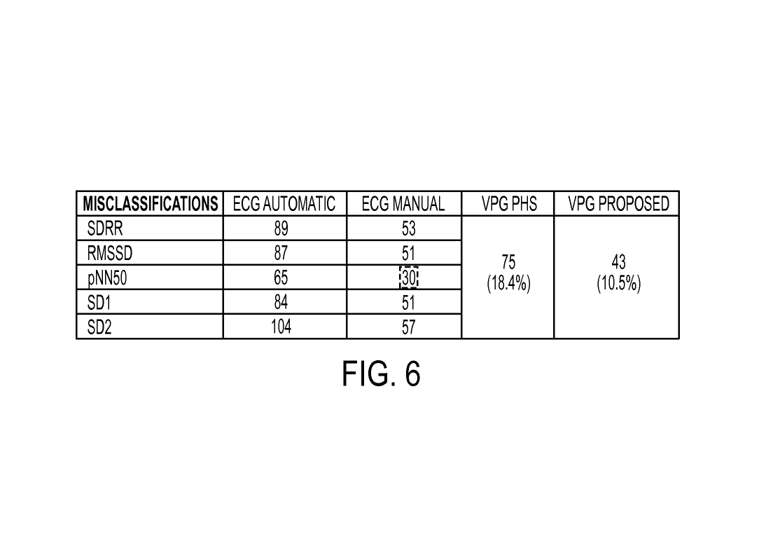

The methods disclosed herein were applied on 11 patients using data obtained through a Xerox-University of Rochester Medical Center (URMC) Research Agreement. Patients with atrial fibrillation (AF) had undergone electrocardioversion to recover back to normal sinus rhythm (SR). The setup included ECG recording via a Holter monitor and video recording with a standard RGB camera at the same time. We were also provided with the manual annotations i.e. whether the beat is normal, AF beat or Ventricular Premature Contraction (VPC), etc. Initially a facial region of interest (ROI) was selected manually from the first video frame. Motion tracking of the ROI was used to automatically select multiple video segments of a duration of 15 seconds with minimum or no motion. These video segments were then pre-processed to extract a VPG signal for the subject. This was followed by calculating the power spectral density (PSD) for all signals. We detected a total of 408 segments [143 (AF)+265 (SR)] from the 11 patients followed by PCA and then training an SVM classifier. ECG RR-intervals and manual annotations for the corresponding video segments were found at the same time. FIG. 6 shows the total misclassifications or error rates during training calculated for both ECG Automatic and ECG Manual using SDRR, RMSSD, pNN50, SD1 and SD2. These are the known metrics to determine the distribution of data and to classify AF from SR. Misclassification numbers under ‘VPG PHS’ were obtained from a video-based VPG signal using pulse harmonic strength (PHS) and ‘VPG Proposed’ are from video but using the present method with a PCA feature set and a SVM classifier. As can be seen from FIG. 6, it can be shown that performance of the classifier was improved by reducing the error rate to 10.5% (43/408) when compared to our previous PHS-based approach which had an error rate of 18.4% (75/408). Also, when compared to ECG—only one metric i.e. pNN50, ECG Manual-annotate had an error rate of 7.3% (30/408).

### Various Embodiments

The teachings hereof can be implemented in hardware or software using any known or later developed systems, structures, devices, and/or software by those skilled in the applicable art without undue experimentation from the functional description provided herein with a general knowledge of the relevant arts. One or more aspects of the methods described herein are intended to be incorporated in an article of manufacture. The article of manufacture may be shipped, sold, leased, or otherwise provided separately either alone or as part of a product suite or a service.

The above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims. The teachings of any publications referenced herein are hereby incorporated by reference in their entirety.

What is claimed is:

1. A method for detecting a cardiac event from a cardiac signal of a subject, the method comprising:
receiving a plurality of different cardiac signals;
transforming each of said cardiac signals to frequency domain signals;
changing said frequency domain signals such that a dominant frequency in each of said signals are substantially aligned to form a matrix of feature vectors;
training a classifier with said matrix of feature vectors;
receiving a cardiac signal of a subject;
transforming said subject's cardiac signal to a frequency domain signal;
changing said subject's frequency domain signal such that a dominant frequency in said signal is substantially aligned with said dominant frequency of said signals used to train said classifier; and
providing said subject's frequency domain signal as a new feature vector to said classifier, said classifier using said new feature vector to classify said subject as having one of: an arrhythmic event, and a non-arrhythmic event.

2. The method of claim 1, wherein said matrix of feature vectors comprises a dimensionality reduced matrix, which is performed using a Principal Component Analysis, said reduced matrix being an eigen feature matrix.

3. The method of claim 1, wherein transforming a cardiac signal to a frequency domain signal comprises performing any combination of: a non-parametric spectral density estimation on said cardiac signals, a parametric spectral density estimation on said cardiac signals, Fourier Transform, Wavelet Transform, and a Discrete Cosine Transform.

4. The method of claim 1, wherein, in response to said subject's frequency domain signal having a frequency bandwidth which is different than that of said signals used to train said classifier, changing said subject's frequency domain signal to have a same sampling frequency and signal length as said training signals.

5. The method of claim 1, wherein said classifier is any of: Support Vector Machine (SVM), a neural network, a Bayesian network, a Logistic regression, Naïve Bayes, Randomized Forests, Decision Trees and Boosted Decision Trees, K-nearest neighbor, a Restricted Boltzmann Machine (RBM), and a hybrid system comprising any combination hereof.

6. The method of claim 1, wherein said cardiac signals are any of: an electrocardiographic signal from an electrocardiographic device, a ballistocardiographic signal from a ballistocardiographic device, an electroencephalographic signal from an electroencephalographic device, an echocardiographic signal from an echocardiographic device, an electromyographic signal from an electromyographic device, a phonocardiographic signal from a phonocardiographic device, and a videoplethysmographic signal from a video imaging device.

7. The method of claim 6, wherein said video imaging device is any of: a contact-based video camera, a non-contact-based video camera, a RGB camera, a multi-spectral camera, a hyperspectral camera, and a hybrid camera comprising any combination hereof.

8. The method of claim 1, wherein, in advance of using said classifier, grouping feature vectors which are positive for an arrhythmic event into a first matrix of feature vectors, and grouping feature vectors which are negative for an arrhythmic event into a second matrix of feature vectors, said classifier being trained using said first and second matrices.

9. The method of claim 1, further comprising a medical professional confirming said subject's classification.

10. The method of claim 9, wherein, in response to said subject's classification being confirmed, further comprising adding said subject's feature vector to said matrix of feature vectors, and updating said classifier.

11. The method of claim 1, wherein, in response to said subject having an arrhythmic event, performing any of: initiating an alert, and signaling a medical professional.

12. The method of claim 1, further comprising communicating said subject's classification to any of: a memory, a storage device, a display device, a handheld wireless device, a handheld cellular device, and a remote device over a network.

13. The method of claim 1, wherein said classification occurs in real-time.

14. A system for detecting a cardiac event from a cardiac signal of a subject, the system comprising:
a classifier; and
a processor in communication with a memory and said classifier, said processor executing machine readable instructions for performing:
retrieving said plurality of different cardiac signals;
transforming each of said cardiac signals to frequency domain signals;
changing said frequency domain signals such that a dominant frequency in each of said signals are substantially aligned to form a matrix of feature vectors;
training said classifier with said matrix of feature vectors;
receiving a cardiac signal of a subject;
transforming said subject's cardiac signal to a frequency domain signal;
changing said subject's frequency domain signal such that a dominant frequency in said signal is substantially aligned with said dominant frequency of said signals used to train said classifier; and
providing said subject's frequency domain signal as a new feature vector to said classifier, said classifier using said new feature vector to classify said subject as having one of: an arrhythmic event, and a non-arrhythmic event.

15. The system of claim 14, wherein said matrix of feature vectors comprises a dimensionality reduced matrix, which is performed using a Principal Component Analysis, said reduced matrix being an eigen feature matrix.

16. The system of claim 14, wherein transforming a cardiac signal to a frequency domain signal comprises performing any combination of: a non-parametric spectral density estimation on said cardiac signals, a parametric spectral density estimation on said cardiac signals, Fourier Transform, Wavelet Transform, and a Discrete Cosine Transform.

17. The system of claim 14, wherein, in response to said subject's frequency domain signal having a frequency bandwidth which is different than that of said signals used to train said classifier, changing said subject's frequency domain signal to have a same sampling frequency and signal length as said training signals.

18. The system of claim 14, wherein said classifier is any of: Support Vector Machine (SVM), a neural network, a Bayesian network, a Logistic regression, Naïve Bayes, Randomized Forests, Decision Trees and Boosted Decision Trees, K-nearest neighbor, a Restricted Boltzmann Machine (RBM), and a hybrid system comprising any combination hereof.

19. The system of claim 14, wherein said cardiac signals are any of: an electrocardiographic signal from an electrocardiographic device, a ballistocardiographic signal from a ballistocardiographic device, an electroencephalographic signal from an electroencephalographic device, an echocardiographic signal from an echocardiographic device, an electromyographic signal from an electromyographic device, a phonocardiographic signal from a phonocardiographic device, and a videoplethysmographic signal from a video imaging device.

20. The system of claim 19, wherein said video imaging device is any of: a contact-based video camera, a non-contact-based video camera, a RGB camera, a multi-spectral camera, a hyperspectral camera, and a hybrid camera comprising any combination hereof.

21. The system of claim 14, wherein, in advance of using said classifier, grouping feature vectors which are positive for an arrhythmic event into a first matrix of feature vectors, and grouping feature vectors which are negative for an arrhythmic event into a second matrix of feature vectors, said classifier being trained using said first and second matrices.

22. The system of claim 14, further comprising a medical professional confirming said subject's classification.

23. The system of claim 22, wherein, in response to said subject's classification being confirmed, further comprising adding said subject's feature vector to said matrix of feature vectors, and updating said classifier.

24. The system of claim 14, wherein, in response to said subject having an arrhythmic event, performing any of: initiating an alert, and signaling a medical professional.

25. The system of claim 14, further comprising communicating said subject's classification to any of: a memory, a storage device, a display device, a handheld wireless device, a handheld cellular device, and a remote device over a network.
